(19) United States
(12) Patent Application Publication
(10) Pub. No.: US 2026/0114857 A1
(43) Pub. Date: Apr. 30, 2026

(54) DEVICES, SYSTEMS, AND RELATED METHODS FOR PARTIALLY REUSABLE MEDICAL DEVICES

(71) Applicant: Boston Scientific Scimed, Inc., Maple Grove, MN (US)

(72) Inventors: Ryan V. WALES, Shrewsbury, MA (US); Juan Pablo ORTIZ GARCIA, Heredia (CR); Barry WEITZNER, Great Barrington, MA (US)

(73) Assignee: Boston Scientific Scimed, Inc., Maple Grove, MN (US)

(21) Appl. No.: 19/366,023

(22) Filed: Oct. 22, 2025

Related U.S. Application Data
(60) Provisional application No. 63/712,743, filed on Oct. 28, 2024.

(51) Int. Cl. A61B 10/06; A61B 13/00; A61B 10/02

(52) U.S. Cl. CPC A61B 10/06; A61B 13/00; A61B 2010/0208

(57) ABSTRACT

Medical devices, systems, and related methods described herein may be useful for reusing a portion of a medical device to perform a medical procedure. The medical device including a shaft that includes a tubular member having a shoulder, and a handle that includes a leg having a protrusion. The protrusion of the handle configured to engage with a surface of the shoulder to removably couple the handle to the shaft.

### CROSS-REFERENCE TO RELATED APPLICATIONS

This application claims the benefit of priority of U.S. Provisional Patent Application No. 63/712,743, filed on October 28, 2024, the entirety of which is incorporated herein by reference.

### TECHNICAL FIELD

Various aspects of the disclosure relate generally to devices, systems, and related methods for partially reusable medical devices. Examples of the disclosure relate to medical devices having reusable handles, among other aspects.

### BACKGROUND

Single-use medical devices are disposed after use, resulting in significant medical waste and negative environmental impact. These medical devices often include a handle, a shaft, and an end effector configured to perform a procedure at a target site, such as biopsy procedures including the removal and analysis of a body tissue sample (“biopsy sample”) for medical diagnosis. It would be useful to improve medical devices, systems, and related methods for medical procedures, for example to reduce medical waste and environment impact associated with medical procedures, such as biopsy procedures.

### SUMMARY

Examples of this disclosure relate to devices, systems, and related methods for partially reusable medical devices, among other things. For example, this disclosure describes examples that relate to reusable handle assemblies for endoscopic medical instruments, among other aspects.

According to one aspect, the disclosure provides a medical device including a shaft and a handle. The shaft including a tubular member having a first shoulder and a second shoulder, each of the first shoulder and the second shoulder extending outwardly relative to a central longitudinal axis of the tubular member. The handle including a first leg having a first protrusion and a second leg having a second protrusion. The first protrusion is configured to engage with a first surface of the first shoulder and the second protrusion is configured to engage with a second surface of the second shoulder to removably couple the handle to the shaft.

According to some aspects, each of the first surface and the second surface may include a plurality of teeth. The plurality of teeth of the first surface may be configured to ratchet to engage the first protrusion, and the plurality of teeth of the second surface may be configured to ratchet to engage the second protrusion. Each of the first surface and the second surface may have a curved shape configured to accommodate a curved path of the first protrusion and the second protrusion as they move inward to engage the plurality of teeth. The first shoulder may be diametrically opposite the second shoulder relative to the central longitudinal axis. The shaft may further include a coil, and wherein the tubular member is positioned within a lumen of the coil. A proximal end of the coil may abut the first shoulder and the second shoulder. The shaft may include an actuation wire, wherein a proximal portion of the actuation wire includes a loop that extends proximally of a proximalmost end of the tubular member. The loop may have a wavy or sinusoidal shape. The loop may be configured to receive a finger of a user.

According to some aspects, the handle may include a hemostatic forceps device. The first leg may be coupled to a first finger ring and the second leg may be coupled to a second finger ring. In a configuration in which the handle is removably coupled to the shaft, the first finger ring and the second finger ring may be at a proximal end of the handle. In another configuration in which the handle is removably coupled to the shaft, the first finger ring and the second finger ring may be at a distal end of the handle.

According to another aspect, the disclosure provides a medical device including a handle, and a shaft including a coil having a plurality of windings. A proximal portion of the coil includes (a) at least one gap defined by a spacing between adjacent windings of the plurality of windings or (b) a shoulder that protrudes outwardly from surrounding portions of the coil. The handle including at least one engagement feature configured to engage the gap or the shoulder to removably couple a proximal portion of the coil to the handle.

According to some aspects, the at least one engagement feature of the handle may include a cavity configured to engage the shoulder. The at least one engagement feature may include a pin vice.

According to yet another aspect, the disclosure provides a medical device including a shaft having an actuation wire, and a handle. The handle including a body having a flat oblong shape, an actuator removably coupled to the body, and a ring that is configured to be removably coupled to a proximal end of the body. The actuator is configured to removably couple to a proximal end of the actuation wire, wherein movement of the actuator relative to the body actuates the actuation wire.

According to some aspects, the body may include a tongue depressor, and the ring may include a spike and a cam member to removably couple the ring to the tongue depressor.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and, together with the description, serve to explain the principles of the disclosure.

FIG. 1 depicts a medical device, according to aspects of the disclosure.

FIG. 2 depicts a portion of a shaft of a medical device, according to aspects of the disclosure.

FIGS. 3A-3B depict portions of a shaft and a handle of a medical device, according to aspects of the disclosure.

FIGS. 4A-4B depict portions of another shaft and another handle, according to aspects of the disclosure.

FIGS. 5A-5B depict portions of another medical device, according to aspects of the disclosure.

FIG. 6 depicts aspects of a handle of a medical device, according to aspects of the disclosure.

FIG. 7 depicts aspects of a handle of another medical device, according to aspects of the disclosure.

### DETAILED DESCRIPTION

Examples of the disclosure include devices, systems, and related methods for medical instruments that can be disassembled after use so that different portions may be disposed or reused according to different protocols.

As used herein, the term “distal” refers to a portion farthest away from a user when introducing a device into a patient and the term “proximal” refers to a portion closest to the user when placing the device into the subject. Labeled arrows “P” and “D” indicate proximal and distal directions, respectively. The terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/- 10% of a stated value.

Examples of the disclosure may relate to system, devices, and methods for performing various medical procedures and/or treating portions of the biliary duct, large intestine, small intestine, cecum, esophagus, any other portion of the gastrointestinal tract, and/or any other suitable patient anatomy (collectively referred to herein as a “target site”). The devices disclosed herein may include a combination of disposable and reusable components/devices. Reference will now be made in detail to examples of the disclosure described above and illustrated in the accompanying drawings. Wherever possible, the same reference numbers will be used throughout the drawings to refer to the same or like parts. Unless otherwise specified, aspects of different embodiments may be combined in any suitable manner.

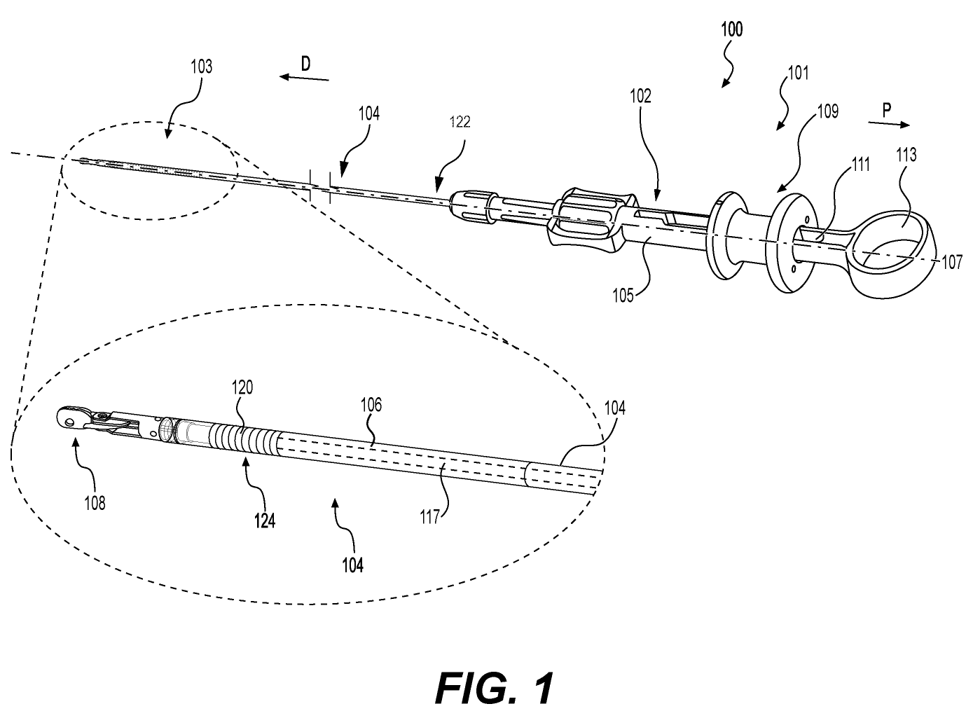

FIG. 1 illustrates a medical device 100 including a proximal portion 101 having a handle 102, and a distal portion 103, according to aspects of the present disclosure. Distal portion 103 may include a shaft 104 extending distally from handle 102 toward an end effector 108. End effector 108 may be positioned at a distal end of shaft 104, e.g., at a distalmost end of distal portion 103. As shown in FIG. 1, end effector 108 may include a pair of jaws for, for example, extracting a biopsy sample of tissue from the target site. However, end effector 108 may be any suitable type of instrument (e.g., snare, stent delivery system, basket, balloon, knife, needle, clip, forceps, suture device, etc.). Distal portion 103 may be delivered through a working channel or other lumen of an endoscope, duodenoscope, gastroscope, colonoscope, ureteroscope, bronchoscope, and/or other medical delivery systems to a target site. Thus, medical device 100 may be an endoscopic instrument (e.g., an endoscopic accessory device).

Shaft 104 may include or otherwise radially surround a wire, for example, a pull wire or an actuation wire 117 extending distally from handle 102 and through shaft 104. Wire 117 may be movable (e.g., within handle 102 and shaft 104) via manipulation of one or more portions of handle 102, for example one or more actuators 109 secured to a proximal end of wire 117. End effector 108 may be coupled (e.g., directly or indirectly) to the distal end of the wire(s). In these aspects, manipulation of actuator 109 may control one or more aspects of medical device 100, for example, control one or more aspects of distal portion such as end effector 108.

As shown, handle 102 includes a body 105 extending along a central longitudinal axis 107 of medical device 100. Body 105 may include one or more inner surfaces defining a lumen or channel of handle 102 which may, for example, be configured to receive or otherwise be coupled (e.g., directly or indirectly) to shaft 104, as described in the examples below. Shaft 104 may be removably coupled to handle 102 using any of the aspects described with respect to the Figures discussed below.

Handle 102 may also include one or more movable members, for example actuator 109. Actuator 109 may be a spool, for example including an indented portion (e.g., with a relatively smaller lateral cross-section) and one or more (e.g., two) ridged or extended portions (e.g., flanges with a relatively larger lateral cross-section). In these aspects, the indented portion may receive one or more of a user’s fingers, such that movement of the user’s finger(s) controls the movement of actuator 109. Actuator 109 may be fixedly coupled to the actuation wire that controls end effector 108 or other aspects of distal portion. Although a spool is shown in FIG. 1, actuator 109 may alternatively be another type of actuator (e.g., finger rings, knob, lever, slider, joystick, etc.).

Body 105 may include a slot 111, for example, extending longitudinally through a portion of body 105, for example, from a position spaced distally from a ring. For example, handle 102 may also include a proximal ring 113 for receiving, for example, a thumb of a user’s hand, while the user’s fingers engage actuator 109. Ring 113 may have any of the features of such rings known in the art. A portion of actuator 109 may extend into a portion of slot 111, such that actuator 109 is movable (e.g., longitudinally movable distally and/or proximally) along slot 111. In these aspects, slot 111 may define a range of movement for actuator 109. Actuator 109 may be movable within slot 111, for example, to control one or more aspects of distal portion, such as to control end effector 108.

It should be understood that actuator 109 may be formed in a variety of sizes to accommodate users with different sized hands or fingers and/or to accommodate different needs of different users. In some examples, handle 102 includes two or more portions coupled together to define actuator 109. For example, actuator 109 (e.g., spool) may be split longitudinally into a first portion and a second portion, which are coupled together at complementary surfaces to form actuator 109. In other examples handle 102 may be unitary or integrally formed, for example by using one or more additive manufacturing and/or subtractive manufacturing processes to define the shape and dimensions of handle 102 and components thereof (e.g., body 105, actuator 109, etc.). Handle 102 may also include a ring 113 (e.g., thumb ring), for example, at a proximal end of body 105. Ring 113 may accommodate a thumb of a user’s hand when the user engages actuator 109.

In some aspects, handle 102 may be formed from a compressed cardboard material, instead of a conventional plastic material. In other aspects, handle 102 may be formed from stainless steel (e.g., LE sintered stainless steel) or other metallic materials that are suitable for autoclaving or other types of sterilization. In aspects, a user may design and additively manufacture (e.g., 3D print) a handle that is tailored to needs (e.g., ergonomic needs) of a user. For example, a practitioner with smaller hands may desire a smaller handle 102. For example, size of ring 113, a length of body 105, or a length of slot 111 (corresponding to a throw of handle 102) may be customized by a user. Furthermore, cushioning may be added to ring 113, actuator 109, or other elements. Handle 102 may have colorings or markings indicating a user with which handle 102 is associated.

In some aspects, shaft 104 may include a sheath 106 having an outer wall defining a lumen extending therein. Sheath 106 may include a non-metallic material, such as plastic (e.g., thermoplastic elastomer (TPE) or thermoplastic polyurethane (TPU), or thermoplastic polyesters), polyamide (e.g., nylon or Pebax), polytetrafluoroethylene (PTFE), or any other suitable non-metal (e.g., insulating) material. A distalmost end of sheath 106 may be directly adjacent to end effector 108.

Shaft 104 may include one or more elements extending through the lumen of sheath 106, such as an actuation wire 117 or a coil 120 having a distal portion 124 coupled to end effector 108 and a proximal portion 122 coupled to (e.g., removably coupled to) handle 102. For example, coil 120 may be disposed in the lumen of sheath 106. Sheath 106 may be directly abutting and contacting coil 120. Coil 120 may be made of a metallic material (e.g., stainless steel, Nitinol, or any other suitable material) and may comprise a wire (having a round or other cross-sectional shape). Coil 120 may have been formed by winding the wire about a mandrel or other structure to form a plurality of windings. Adjacent windings of coil 120 may contact one another, so that at least portions of coil 120 have an outer wall without substantial gaps (or substantially without gaps along a longitudinal extent thereof). Coil 120 may be shaped and sized to extend through the lumen of sheath 106, and coil 120 may define a channel or lumen extending along a central axis coaxial or parallel with central axis 107. The channel may be configured to receive wire 117 for, for example, actuating end effector 108.

In some aspects, components of distal portion 103 (e.g., sheath 106, end effector 108, etc.) may include disposable and/or recyclable material, for example to discard after the medical procedure. These components having disposable or recyclable material may be detached from handle 102, separated from other components, and discarded. In other aspects, handle 102 (or components thereof) may be detached from distal portion 103 to, for example, clean and reuse one or more components of handle 102 (e.g., with another shaft 104 for performing another medical procedure). Alternatively, during the course of a procedure, handle 102 may be removed to couple handle 102 to another type of distal portion 103 (e.g., having a different end effector 108) or to a further distal portion 103 of a same type.

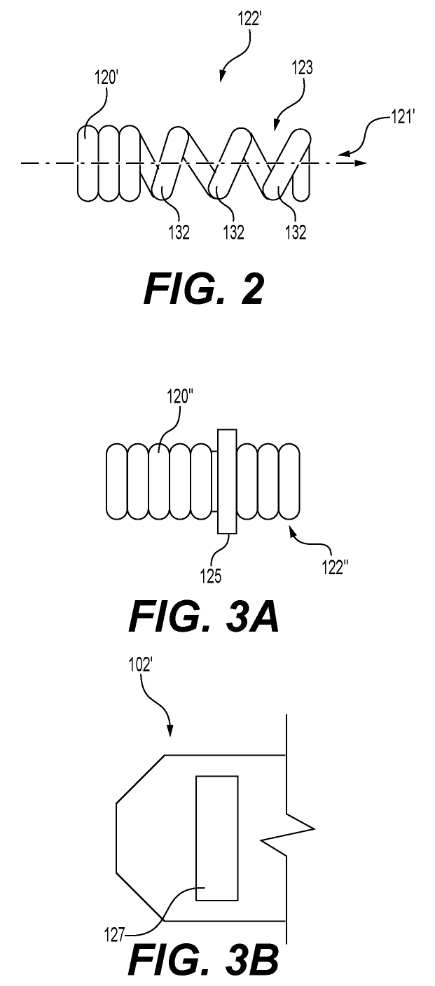

FIG. 2 shows a proximal portion 122’ of a coil 120’, having any of the features of coil 120, unless otherwise stated. Coil 120’ may be at least partially within sheath 106 (not shown in FIG. 2 but shown in FIG. 1). Proximal portion 122’ of coil 120’ may extend proximally of a proximalmost end of sheath 106. Adjacent windings 132 of proximal portion 122’ may be spaced apart from one another so as to form a one or more gaps 123. Gaps 123 may be engagement features for engaging handle 102 to removably couple handle 102 to coil 120.

Handle 102 may include one or more engagement features for engaging with windings 132 and/or gaps 123. For example, handle 102 may include one or more features for threading with proximal portion 122’. For example, handle 102 may include one or more threads or protrusions for engaging with proximal portion 122’. For example, protrusions of handle 102 may extend through gaps 123. In some examples, features of handle 102 extend through gap 123 and into a channel 121’ of coil 120’ defined therethrough (a lumen of coil 120’ defined by windings 132), e.g., to grasp or otherwise couple with proximal portion 122’ and attach disposable coil 120’ to handle 102. For example, gap123 may be shaped and sized to receive a pin vice or the like of handle 102 therein, e.g., for securing handle 102 with coil 120’ to perform the medical procedure. In some examples, the engagement feature of handle 102 have a first end secured to an inner surface of body 105 of handle 102 and a second end (opposite the first end) configured to selectively grasp coil 120’. In some aspects, coil 120’ may be coupled to handle 102 manually (e.g., via a user’s hands with or without using assembly tools). Other engagement features be used to automatically attach handle 102 with coil 120’. Following a procedure, coil 120’ may be detached from handle 102 and disposed of, while handle 102 is reused.

FIG. 3A depicts an alternative coil 120’’, and FIG. 3B depicts a portion (e.g., a distal portion) of an alternative handle 102’. Whereas coil 120’ may couple to handle 102’ via gap 123 on coil 120’ and a protruding portion on handle 102, coil 120’’ may include a protruding shoulder 125 and handle 102’ may include a cavity 127 for engaging with shoulder 125. Shoulder 125 may protrude outwardly relative to surrounding portions of coil 120’. Shoulder 125 may be fixedly secured to a proximal portion 122’’ of coil 120” by, for example, crimping, heat shrinking, etc. A distal portion of coil 120” may extend distally from proximal portion 122” and shoulder 125 (e.g., to end effector 108). Alternatively, shoulder 125 may be releasably secured to proximal portion 122’’ of coil 120’’ via threaded coupling or another mechanism.

To removably couple proximal portion 122’’ to handle 102’, shoulder 125 may be inserted into cavity 127. Shoulder 125 may be removed from cavity 127 after a procedure to uncoupled coil 120’’ from handle 102’, so as to dispose of coil 120’’ and reuse handle 102’. Cavity 127 may be sized and shaped so as to removably accommodated should 125.

In some aspects, handle 102’ may include a pin vice (e.g., in cavity 127). When handle 102’ is coupled to coil 120’’, the pin vice may be aligned with (e.g., may terminate at) shoulder 125. In such examples, shoulder 125 may assist with reliably removably positioning coil 120’’ relative to handle 102’. For example, a distal end of handle 102’ may abut shoulder 125 to position handle 102’ appropriately relative to coil 102’’.

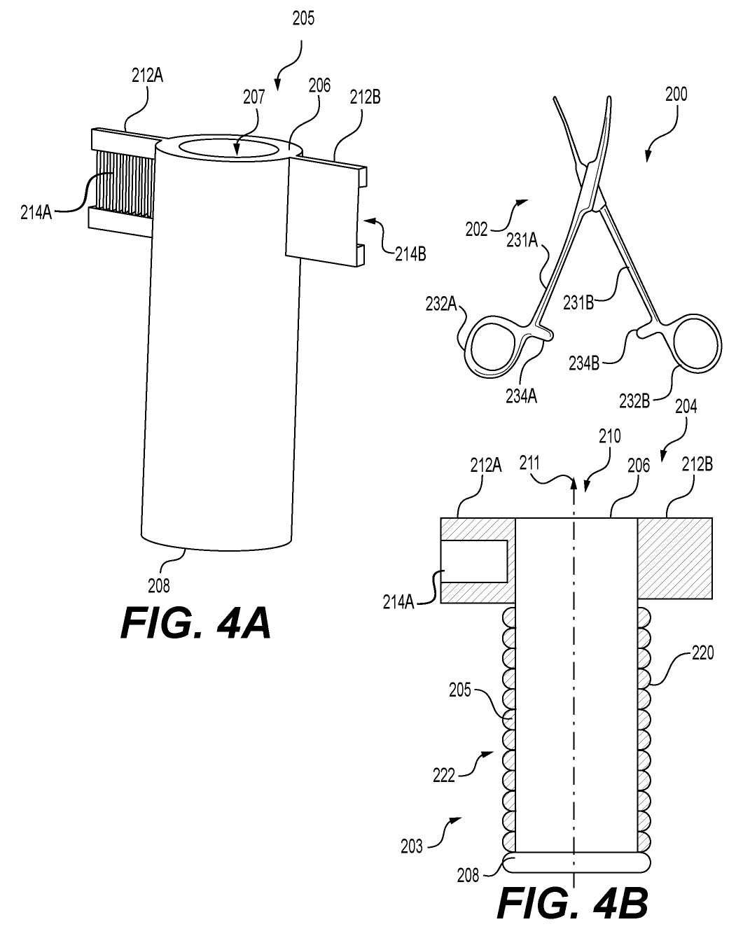

FIGS. 4A-4B illustrate aspects of proximal portions of another medical device 200, according to aspects of the present disclosure. Unless otherwise specified, medical device 200 may include aspects of medical device 100. Where feasible, reference numbers of medical device 200 add 100 to the reference numbers of medical device 100 to indicate similar structures. As shown in FIG. 4B and discussed herein, medical device 200 includes a handle 202 configured to be removably attached to a distal portion 203 of medical device 200 to perform the medical procedure (e.g., biopsy procedure) and be detached after the medical procedure (e.g., to dispose of distal portion). Handle 202 may be a medical tool commonly used in an endoscopic or operating suite, such as a pair of hemostatic biopsy forceps. Handle 202 is not shown to scale with distal portion 203 in FIG. 4B.

Medical device 200 may include distal portion 203 (FIG. 4B) having any of the properties of distal portion 103, unless otherwise specified herein. End effector 108 may be disposed at a distal end (not shown) of distal portion 203. As shown in FIG. 4B, distal portion 203 may include a shaft 204 defining a lumen 210 therein. Shaft 204 may extend along a central longitudinal axis 211. Shaft 204 may include a coil 220, having any of the properties of coil 120.

A proximal end of shaft 204 may include a hypotube 205 (or other tubular member) extending between a first, proximal surface 206 and a second, distal surface 208. A distal portion of hypotube 205 may be disposed within a lumen of coil 220, as shown in FIG. 4B. In other examples, a proximal portion 222 of coil 220 may inserted into a lumen 207 of hypotube 205 via a distal opening formed in second surface 208 of hypotube 205, such that hypotube 205 is positioned circumferentially around proximal portion 222 of coil 220. Lumen 207 may be shaped and sized to receive a wire therethrough, such as wire 117. For example, an actuation wire may extend through lumen 207, such that the actuation wire is movable (proximally and/or distally) through and relative to hypotube 205 and coil 220 (e.g., approximately along central axis 211). For example, medical device 200 may have a wire with any of the features of the wire of device 300, described below.

Shaft 204 also includes one or more engagement elements for, for example, engaging handle 202. As shown in FIG. 2, shaft 204 includes a first shoulder 212A (protrusion) positioned opposite a second shoulder 212B (protrusion) relative to central axis 211. First shoulder 212A and second shoulder 212B (“shoulders 212A-212B”, collectively) each extend radially from the outer wall of hypotube 205 in different directions relative to central axis 211. In the example shown, shoulders 212A-212B are positioned diametrically opposed each other relative to central axis 211; however, shoulders 212A-212B may have different positions relative to each other and central axis 211 along the outer wall of hypotube 205. In some examples, shoulders 212A-212B are integrally formed with hypotube 205, and, in other examples shoulders 212A-212B are secured to the outer surface of hypotube 205 using one or more fixation processes (e.g., crimping, welding, soldering, etc.). A portion of hypotube 205 distal to shoulders 212A and 212B may be disposed within a lumen of coil 220. Shoulders 212A and 212B may rest on or abut a proximal surface of coil 220, as shown in FIG. 4B. Shoulders 212A and 212B may thus retain coil 220 relative to hypotube 205 and handle 202 in a desire position.

Shoulders 212A-212B may engage handle 202 to removably couple handle 202 to shaft 204. As shown, first shoulder 212A includes a first surface 214A having a first plurality of teeth (e.g., ratcheting teeth thereon) thereon, as shown in FIG. 4A. Second shoulder 212B includes a second surface 214B, which may also include teeth thereon. As shown in FIGS. 4A and 4B, second surface 214B may face an opposite direction to first surface 214A (e.g., first surface 214A faces out of the page in FIG. 4B and second surface 214B faces into the page in FIG. 4B). Alternatively, second surface 214B and first surface 214A may face the same direction. Surfaces 214A and 214B may be recessed (inset) with respect to surrounding portions of shoulders 212A and 212B.

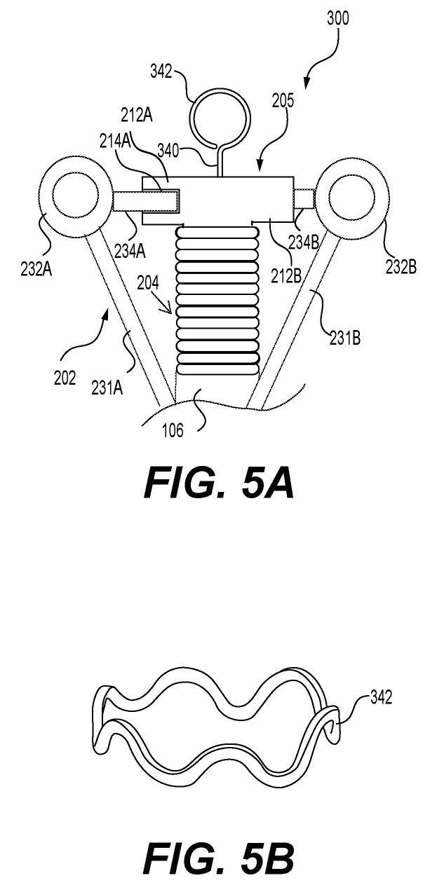

In the example shown, handle 202 is a hemostatic biopsy forceps. Handle 202 may have a pair of legs 231A, 231B. Leg 231A (a first leg) may include (be coupled to) a first ring 232A adjacent a first protrusion 234A. Leg 231B (a second leg) may include (be coupled to) a second ring 232B adjacent a second protrusion 234B. Rings 232A and 232B may be shaped and sized to respectively receive different user fingers therein. Shoulders 212A and 212B may be sized and shaped to engage protrusions 234A and 234B to removably couple handle 202 to shaft 204. For example, first protrusion 234A may engage the first plurality of teeth on first surface 214A, and second protrusion 234B may engage a second plurality of teeth on second surface 214B. For example, each of protrusions 234A and 234B may teeth (e.g., ratcheting teeth), such that protrusions 234A and 234B engage shoulders 212A and 212B, respectively, with a ratcheting action to removably secure handle 102 to shaft 104. In examples, a user may selectively ratchet protrusions 234A and 234B relative to shoulders 212A and 212B to customize a spacing of rings 232A and 232B to accommodate a user’s hand. Although handle 202 is not shown coupled to shaft 204 in FIG. 4B, it will be appreciated that protrusions 234A and 234B may engage shoulders 21A and 212B similarly to the manner shown in FIG. 5A.

In some examples, shoulders 212A and 212B may be angled at a non-90 degree relative to central axis 211 and hypotube 205. In aspects, shoulders 212A and 212B (or surfaces 214A, 214B thereof) may have an arcuate or other curved shape. This may be useful, for example, to accommodate an arcuate path of protrusions 234A and 234B as they move inward toward central axis 211 to engage with shoulders 212A and 212B, respectively.

FIGS. 5A and 5B illustrate aspects of another medical device 300, according to aspects of the present disclosure. Unless otherwise specified, medical device 300 may include any feature of medical device 200. However, handle 202 may be arranged to face an opposite direction from handle 202 in medical device 300. Handle 202 and shaft 204 are not necessarily shown to scale in FIG. 5A. For example, shaft 204 may be narrower/smaller relative to handle 202. In medical device 200, handle 202 may be coupled to shaft 204 such that rings 232A and 232B are a distalmost portion of handle 202 (e.g., rings 232A and 232B face downward and the clamp portion of the hemostat faces upward/proximally). In medical device 300, handle 202 may be coupled to shaft 204 such that rings 232A and 232B are a proximalmost portion of handle 202 (e.g., rings 232A and 232B face upward and the clamp portion of the hemostat faces downward/distally). A user may select a position of handle 202 between the configurations of medical devices 200 and 300 to suit the user’s preferences and/or ergonomic needs. In some examples, shoulders 212A and 212B may have an arcuate or other curved shape or may have surfaces 214A and 214B with an arcuate shape or other curved shape that is different from an arcuate shape of medical device 200, to accommodate a different curved path of protrusions 234A, 234B.

As shown in FIG. 5A, a wire 340 may extend through lumen 210 and lumen 207 (lumen 207 and lumen 210 are shown in FIGS. 4A and 4B, respectively). Wire 340 is movable through and relative to lumen 210 and lumen 207. A distal portion of wire 340 may be coupled to end effector 108 to actuate end effector 108. Wire 340 may have any of the features of wire 117, unless otherwise specified. A loop 342 may be secured to a proximalmost end of wire 340. Loop 342 may be proximal of a proximalmost end of shaft 204, including hypotube 205. Loop 342 may be a braided wire loop. In some implementations, loop 342 is formed of a same material as wire 340, such that loop 342 is integrally formed with wire 340. In other implementations, loop 342 may be separately formed and crimped or otherwise coupled to the proximal portion of wire 340.

Loop 342 may be configured to receive a user’s finger, so that a user may pull or push loop 342 to actuate wire 340. Loop 342 may be shaped and dimensioned to prevent injury of user’s fingers (e.g., may have an enlarged diameter). For example, as shown in FIG. 5B, loop 342 may have a wavy shape or a zig-zag shape (e.g., a sinusoidal shape), which may be useful to increase surface area in contact with the user’s finger(s) and to facilitate actuating wire 340. In alternatives, loop 342 may be omitted, and a proximal end of wire 340 may be covered by a hypotube that has a larger diameter than lumen 210/lumen 207. In further alternatives, wire 340 may include both loop 342 and a hypotube that is distal to loop 342 in order to provide for increased stiffness of wire 340.

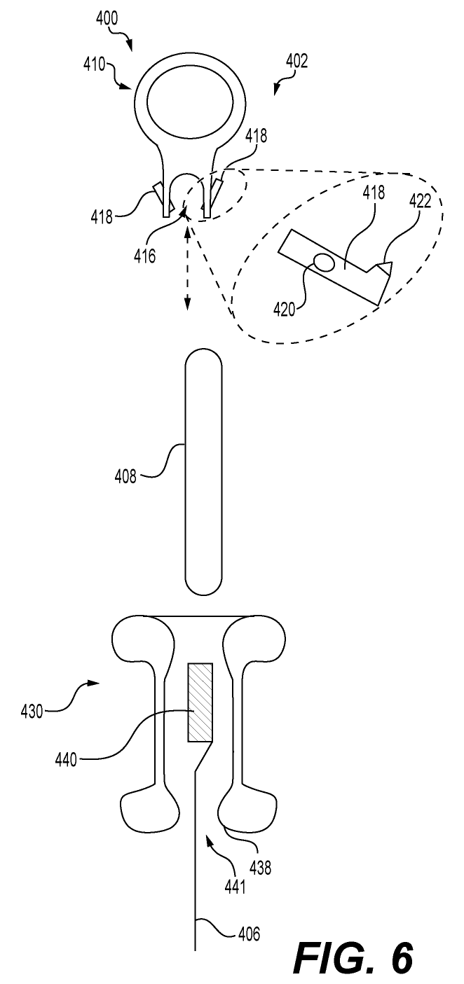

FIG. 6 illustrates portions of another medical device 400, according to aspects of the present disclosure. Unless otherwise specified, medical device 400 may include one or more aspects of medical devices 100, 200, and/or 300. In the example shown, medical device 400 includes a proximal portion having a handle 402 configured to be attached/detached with a distal portion of medical device 400, e.g., for performing a medical procedure. The distal portion may have disposable components, e.g., for discarding and/or recycling after the medical procedure. Although the distal portion of medical device 400 is not shown, the distal portion may have any of the features of any of the shafts/distal portions described above (e.g., shafts, coils, end effectors, etc.).

Handle 402 may have a disposable body 408. In some aspects, disposable body 408 may be a tongue depressor, which may be commonly available. Disposable body 408 may be discarded or disposed of by the user after the medical procedure. Body 408 may have a flat oblong shape extending between proximal and distal ends of body 408. The shaft (e.g., coil) of medical device 400 may be removably and fixedly coupled to body 408 by any of the mechanisms disclosed herein as to other Figures.

Handle 402 may also include an actuator 430 that is removably attachable to body 408. Actuator 430 may be removably or fixedly coupled to a proximal portion of a wire 406, having any of the properties of wires 117 or 340. Actuator 430 may have any of the properties of actuator 109, unless otherwise provided. In examples, actuator 430 may be a spool. Actuator 430 may include a body/housing 440 configured to secure the proximal portion of wire 406 therein. For example, housing 440 may define a lumen 441 having a tortuous shape to removably secure wire 406 within housing 440. In examples, wire 406 may include a widened proximal portion (e.g., a hypotube) to help to secure wire 406 within housing 440.

Actuator 430 may also be removably coupled to body 408. For example, actuator 430 may include two pieces that fit together around body 408. In examples, actuator 430 may have a clamshell shape that is able to be opened and closed to couple actuator 109 to body 408. When actuator 430 is coupled to body 408, proximal or distal movement of actuator 430 along body 408 may actuate wire 406 proximally and distally, thereby actuating end effector 108 (FIG. 1). Actuator 430 may have a surface contoured for complementary engagement with disposable body 408. For example, actuator 430 may have a curved surface 438 that reduces friction between actuator 430 and disposable body 408 during movement of actuator 430 and wire 406. The proximal portion of wire 406 may extend along a side surface (e.g., a planar surface) of body 408 when actuator 430 is coupled to body 408.

In some aspects, handle 402 may include a proximal ring 410 to receive a thumb of a user, similar to ring 113 of handle 102. A user may insert a thumb into ring 410 while engaging actuator 430 in order to hold handle 102. Ring 410 may define a cavity 416 therein that is shaped and sized to receive a proximal end of body 408. Ring 410 may have engagement features configured to secure disposable body 408 within cavity 416. For example, the engagement features may include set screw, vice pin, cam or the like.

In the example shown, ring 410 includes a cam 418 (cam member), which may be movable between a first position (e.g., open position) and a second position (e.g., closed position) to selectively secure disposable body 408 within cavity 416. In the first position, cam 418 may be disposed at least partially outside of cavity 416, and moving cam 418 toward the second position causes cam 418 to engage disposable body 408 positioned within cavity 416. In some examples, cam 418 may include a spring loaded cam having one or more biasing elements, for example one or more springs which bias cam 418 toward the closed position. The biasing elements (e.g., springs) may apply a spring force on cam 418 to maintain the closed position (e.g., and engagement with disposable body 408). To remove ring 410 following a procedure, the medical professional may press a button or the like to move cam 418 from the closed position toward the open position, for example about a pivot 420 pivotably coupling cam 418 with ring 410. Pivot 420 may include one or more pins extending through one or more complementary openings formed in cam 418 and formed in ring 410.

Cam 418 (or another portion of ring 410) may include a barb, spike, or other sharp element that is configured to puncture or otherwise engage disposable body 408, e.g., to penetrate and secure disposable body 408 within cavity 416. In the example shown in FIG. 6, cam 418 includes a spike 422 that is shaped and contoured to penetrate an outer surface of disposable body 408, e.g., when moving from the open position into the closed position of cam 418. Disposable body 408 may include wood or other material that is disposable and/or recyclable (e.g., after the medical procedure). In other implementations, another recyclable material may be utilized to form the handle body (e.g., to replace disposable body 408), such as compressed cardboard or other material.

Before a medical procedure, a user may couple wire 406 of a disposable distal portion of medical device 400 to actuator 430. The user may couple actuator 430 to body 408. The user may couple ring 410 to body 408. The user may then perform a medical procedure with device 400. Following the procedure, the user may disconnect the ring 410 and actuator 430 from body 408. The user may disconnect actuator 430 from wire 406. The user or another professional may dispose of body 408 and the distal portion (including wire 406). Actuator 430 and ring 410 may be reused in a subsequent procedure.

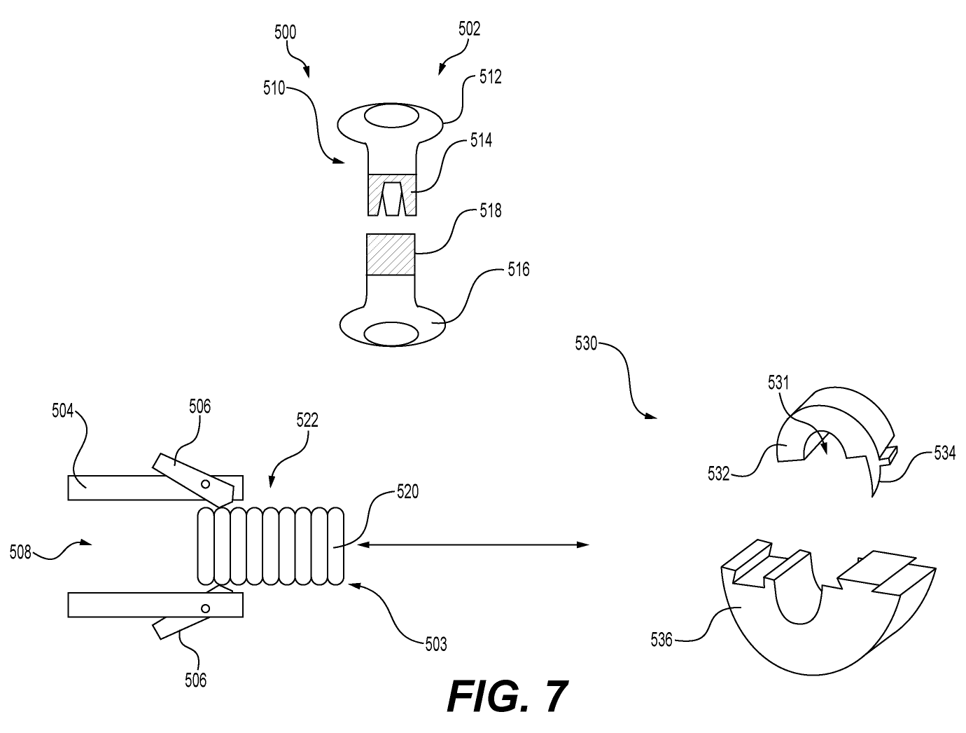

FIG. 7 illustrates another exemplary medical device 500, including a handle 502 and a disposable distal portion 503 according to aspects of the disclosure. Unless otherwise specified, medical device 500 may include one or more aspects of medical devices 100, 200, 300, and/or 400. For example, handle 502 may be similar to handle 102 but may be configured for assembly and/or for disassembly via hand or tool in order to reuse handle 502 and dispose of distal portion 503.

Handle 502 may include one or more movable actuators, such as a actuator 530, having any of the properties of, for example, actuators 109 or 430. Actuator 530 may be longitudinally movable distally and/or proximally along a longitudinal axis of medical device 500. Actuator 530 may be formed by a first spool component 532 and a second spool component 536. Actuator 530 may be divided approximately along a plane including longitudinal axis of medical device 500, for example, to form first spool component 532 and second spool component 536. When assembled, respective portions of each of first spool component 532 and second spool component 536 may collectively define a cavity 531 of actuator 530. Cavity 531 may be sized and/or shaped to receive proximal portion of an actuation wire therein (having any of the properties of the actuation wires described herein). For example, the actuation wire may include a crimp, ferrule, or other widened feature configured to engage cavity 531 and to removably retain the actuation wire to actuator 530.

In some aspects, medical device 500 may include a pin vice 510, including a first vice component 512 having first mating feature 514 and a second vice component 516 having second mating feature 518. First vice component 512 and second vice component 516 may assemble to form pin vice 510 (e.g., via mating features 514, 518). Pin vice 510 may be assembled, for example, around one or more actuation wires disposed therein. First vice component 512 and second vice component 516 may define a cavity shaped and sized to receive the actuation wire(s) therethrough. In some examples, as shown, first and second vice components 512, 516 assemble via threaded engagement. However other engagement means are contemplated herein, for example compression fit or snap fit means for securing first and second vice components 512, 516 together to form pin vice 510. In some aspects, pin vice 510 may be fixedly coupled to (e.g., integrally formed with) other portions of actuator 530. For example, one of vice components 512, 516 may be fixedly coupled to one of spool components 532, 536, and the other of vice components 512, 516 may be fixedly coupled to with the other of spool components 532, 536. In other aspects, pin vice 510 may be inserted to and removed from a remainder of actuator 530.

In some aspects, first spool component 532 and second spool component 536 may releasably engage (e.g., surround) body 504 of handle 502. Actuator 530 may include a clipping feature 534. In some examples, actuator 530 may include a living hinge feature with a user interface, releasable component, slidable feature or other mechanism for coupling actuator 530 to body 504 without tools before a medical procedure and for removal of actuator 530 following a medical procedure. In some aspects, actuator 530 may be non-removably coupled to body 504 but may hingedly open to reveal cavity 531 to removably couple the actuation wire to actuator 530. Proximal and distal movement of actuator 530 relative to body 504 may actuate end effector 108 (not shown) by moving the actuation wire proximally and distally.

Body 504 may define a channel 508 therein. Channel 508 may have an opening on a distal end of body 504. Handle 502 may include a coil retention feature on an inner surface of body 504. The coil retention feature may releasably secure a proximal portion of a coil 520 at least partially received within channel 508. For example, body 504 may include a pin vice, living hinge, cam(s) or the like configured to selectively attach the proximal portion of coil 520 with the body 504.

In the example shown, body 504 includes a cam 506 positioned at the distal portion thereof and configured to selectively engage coil 520, for example to secure proximal portion 522 of coil 520 within channel 508. Cam 506 may move at least between an open position (e.g., to receive coil 520 within channel 508) and a closed position (e.g., to secure proximal portion 522 therein). For example, cam 506 may include two arms that may be moved proximally to release coil 520 and distally to secure coil 520. As shown, coil 520 is shaped and dimensioned for insertion into body 504 via the opening and, once proximal portion 522 is inserted into channel 508, the medical professional may move cam 506 from the open position toward the closed position to engage proximal portion 522 and secure coil 520 to body 504.

In some examples, cam 506 may include a latch or other mechanism configured to be manually movable (e.g., by the medical professional and into engagement with coil 520). In other examples, cam 506 may include a biasing mechanism, such as one or more springs, configured to bias cam 506 toward at least one of the open position or closed position (e.g., for example into engagement with proximal portion 522 of coil 520 received therein). In some aspects, these features support reusing handle 502, for example by attaching one disposable portion (e.g., coil 520) to perform one medical procedure with handle 502, and attaching another disposable portion (e.g., another coil 520) to perform another medical procedure using handle 502.

Medical devices, systems, and related methods discussed herein may be useful for performing biopsy medical procedures to acquire a biopsy sample. Each of the aforementioned devices, systems, and methods may be used for medical procedures to acquire a biopsy sample from a patient. By providing a medical device capable of acquiring biopsy samples using a reusable handle, known problems associated with biopsy medical procedures and/or other aspects of invasive surgical procedures may be reduced or avoided. Based on these aspects, physicians or other users of may reduce the overall procedure time, increase efficiency of procedures, and/or avoid unnecessary harm to patient anatomy during procedures that involve acquiring biopsy samples.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device, comprising:
a shaft including a tubular member having a first shoulder and a second shoulder, each of the first shoulder and the second shoulder extending outwardly relative to a central longitudinal axis of the tubular member; and
a handle including a first leg having a first protrusion and a second leg having a second protrusion;
wherein the first protrusion is configured to engage with a first surface of the first shoulder and the second protrusion is configured to engage with a second surface of the second shoulder to removably couple the handle to the shaft.

2. The medical device of claim 1, wherein each of the first surface and the second surface includes a plurality of teeth.

3. The medical device of claim 2, wherein the plurality of teeth of the first surface are configured to ratchet to engage the first protrusion, and wherein the plurality of teeth of the second surface are configured to ratchet to engage the second protrusion.

4. The medical device of claim 2, wherein each of the first surface and the second surface has a curved shape configured to accommodate a curved path of the first protrusion and the second protrusion as they move inward to engage the plurality of teeth.

5. The medical device of claim 1, wherein the handle includes a hemostatic forceps device.

6. The medical device of claim 1, wherein the first surface faces a first direction, and wherein the second surface faces a second direction, opposite to the first direction.

7. The medical device of claim 1, wherein the first shoulder is diametrically opposite the second shoulder relative to the central longitudinal axis.

8. The medical device of claim 1, wherein the shaft further includes a coil, and wherein the tubular member is positioned within a lumen of the coil.

9. The medical device of claim 8, wherein a proximal end of the coil abuts the first shoulder and the second shoulder.

10. The medical device of claim 1, wherein the first leg is coupled to a first finger ring and the second leg is coupled to a second finger ring.

11. The medical device of claim 10, wherein, in a configuration in which the handle is removably coupled to the shaft, the first finger ring and the second finger ring are at a proximal end of the handle.

12. The medical device of claim 10, wherein, in a configuration in which the handle is removably coupled to the shaft, the first finger ring and the second finger ring are at a distal end of the handle.

13. The medical device of claim 1, wherein the shaft includes an actuation wire, wherein a proximal portion of the actuation wire includes a loop that extends proximally of a proximalmost end of the tubular member.

14. The medical device of claim 13, wherein the loop has a wavy or sinusoidal shape.

15. The medical device of claim 13, wherein the loop is configured to receive a finger of a user.

16. A medical device, comprising:
a shaft including a coil having a plurality of windings, wherein a proximal portion of the coil includes (a) at least one gap defined by a spacing between adjacent windings of the plurality of windings or (b) a shoulder that protrudes outwardly from surrounding portions of the coil; and
a handle including at least one engagement feature configured to engage the gap or the shoulder to removably couple a proximal portion of the coil to the handle.

17. The medical device of claim 16, wherein the at least one engagement feature includes a cavity configured to engage the shoulder.

18. The medical device of claim 16, wherein the at least one engagement feature includes a pin vice.

19. A medical device, comprising:
a shaft having an actuation wire; and
a handle including:
a body having a flat oblong shape;
an actuator removably coupled to the body, wherein the actuator is configured to removably couple to a proximal end of the actuation wire, wherein movement of the actuator relative to the body actuates the actuation wire; and
a ring that is configured to be removably coupled to a proximal end of the body.

20. The medical device of claim 19, wherein the body is a tongue depressor, and wherein the ring includes a spike and a cam member to removably couple the ring to the tongue depressor.
